(19) United States
(12) Patent Application Publication
(10) Pub. No.: US 2023/0339109 A1
(43) Pub. Date: Oct. 26, 2023

(54) AUTOMATIC SELECTION OF COLLABORATIVE ROBOT CONTROL PARAMETERS BASED ON TOOL AND USER INTERACTION FORCE

(71) Applicant: KONINKLIJKE PHILIPS N.V., EINDHOVEN (NL)

(72) Inventor: MARCIN ARKADIUSZ BALICKI, CAMBRIDGE, MA (US)

(21) Appl. No.: 18/009,491

(22) PCT Filed: Jun. 10, 2021
(86) PCT No.: PCT/EP2021/065550

§ 371 Date: Dec. 9, 2022

Related U.S. Application Data
(60) Provisional application No. 63/038,149, filed on Jun. 12, 2020.

(51) Int. Cl. B25J 9/16; B25J 13/08; A61B 34/30; A61B 17/17

(52) U.S. Cl. CPC B25J 9/1633; B25J 13/085; B25J 9/161; A61B 34/30; A61B 17/17; G05B 2219/39001; G05B 2219/32335

(57) ABSTRACT

A system includes: a robotic arm which has an instrument interface; a force/torque sensor for sensing forces at the instrument interface; a robot controller for controlling the robotic arm and to control a robot control parameter; and a system controller. The system controller: receives temporal force/torque data, wherein the temporal force/torque data represents the forces at the instrument interface over time during a collaborative procedure with a user; analyzes the temporal force/torque data to determine a current intention of the user and/or a state of the collaborative procedure; and causes the robot controller to control the robotic arm in a control mode which is predefined for the determined current intention of the user or state of the collaborative procedure, wherein the control mode determines the robot control parameter.

### TECHNICAL FIELD

This invention pertains to robots, and particularly collaborative robots, which may be employed, for example, in surgical operating rooms, and methods of operating such collaborative robots.

### BACKGROUND AND SUMMARY

Collaborative robots are robots that work in the same space as humans, often directly interacting with humans, for example through force control. An example of such a collaborative robot is a robot which includes an end effector for holding a tool, or a tool guide for a tool, while a human manipulates the tool to accomplish a task. Collaborative robots are generally considered to be safe and don't require specialized safety barriers. With growing acceptance of such collaborative robots, humans are expecting more intelligent and automatic behaviors from these collaborative robots.

Collaborative robots should have advanced perception to provide intuitive assistance in protocol-heavy workflows, such as those in surgical operating rooms. However, compared to humans, robots have very poor context perception due to limited sensing modalities, quality, and bandwidth feedback. To make these robots truly collaborative they require some ability to autonomously change their behavior based on the state of the environment, the task at hand, and/or the user's intention.

Accordingly, it would be desirable to provide collaborative robot and a method of operating a collaborative robot. In particular it would be desirable to provide such a collaborative robot and a method of operating a collaborative robot which may provide automatic selection of one or more robot control parameters based on the state of the environment, the task at hand, and/or the user's intention.

In one aspect of the invention, a system comprises: a robotic arm having one or more degrees of freedom of control, wherein the robotic arm includes an instrument interface; at least one force/torque sensor configured to sense forces at the instrument interface; a robot controller configured to control the robotic arm to move the instrument interface to a determined position and to control at least one robot control parameter; and a system controller. The system controller is configured to: receive temporal force/torque data, wherein the temporal force/torque data represents the forces at the instrument interface over time, sensed by the at least one force/torque sensor, during a collaborative procedure with a user, analyze the temporal force/torque data to determine at least one of a current intention of the user and a state of the collaborative procedure, and cause the robot controller to control the robotic arm in a control mode which is predefined for the determined current intention of the user or state of the collaborative procedure, wherein the control mode determines at least one robot control parameter.

In some embodiments, the instrument interface comprises a tool guide which is configured to be interfaced with a tool which can be manipulated by the user during the collaborative procedure, and the forces applied by the user to the instrument interface comprise at least one of: forces applied indirectly to the tool guide during user manipulation of the tool; (2) forces applied directly to the tool guide by the user; (3) forces from an environment of the robot; and (4) forces generated by the tool.

In some embodiments, the system controller is configured to apply the temporal force/torque data to a neural network to determine the current intention of the user or the state of the collaborative procedure.

In some embodiments, the neural network is configured to determine from the temporal force/torque data when the user is drilling with the tool, and is further configured to determine from the temporal force/torque data when the user is hammering with the tool.

In some embodiments, the at least one robot control parameter controls a rendered stiffness of the tool guide against the forces applied in at least one direction.

In some embodiments, when the neural network determines from the temporal force/torque data that the user is hammering on the tool, the neural network further determines whether the tool is hammering through bone or is hammering through tissue, wherein when the tool is determined to be hammering through soft tissue, the control mode is a first stiffness mode wherein the robot controller controls the tool guide to have a first stiffness, and wherein when the tool is determined to be hammering through bone, the control mode is a second stiffness mode wherein the robot controller controls the tool guide to have a second stiffness, wherein the second stiffness is less than the first stiffness.

In some embodiments, the system provides an alert to the user when the system changes the control mode.

In some embodiments, the system controller is further configured to receive auxiliary data comprising at least one of video data, image data, audio data, surgical plan data, diagnostic plan data and robot vibration data, and is still further configured to determine the current intention of the user or the state of the collaborative procedure based on the temporal force/torque data and the auxiliary data.

In another aspect of the invention, a method is provided for operating a robotic arm having one or more degrees of freedom of control, wherein the robotic arm includes an instrument interface. The method comprises: receiving temporal force/torque data, wherein the temporal force/torque data represents forces at the instrument interface over time, sensed by a force/torque sensor during a collaborative procedure with a user; analyzing the temporal force/torque data to determine at least one of a current intention of the user and a state of the collaborative procedure; and controlling the robotic arm in a control mode which is predefined for the determined current intention of the user or state of the collaborative procedure, wherein the control mode determines at least one robot control parameter.

In some embodiments, the instrument interface comprises a tool guide which is configured to be interfaced with a tool which can be manipulated by the user during the collaborative procedure, and wherein the force/torque sensor measures at least one of: (1) forces exerted indirectly on the tool guide by the user during user manipulation of the tool; (2) forces applied directly to the tool guide by the user; (3) forces from an environment of the robot; and (4) forces generated by the tool.

In some embodiments, analyzing the temporal force/torque data to determine at least one of the current intention of the user and the state of the collaborative procedure comprises applying the temporal force/torque data to a neural network to determine the current intention of the user or the state of the collaborative procedure.

In some embodiments, the neural network determines from the temporal force/torque data when the user is drilling with the tool, and further determines from the temporal force/torque data when the user is hammering with the tool.

In some embodiments, the at least one robot control parameter controls a rendered stiffness of the tool guide against the forces applied in at least one direction.

In some embodiments, when the neural network determines from the temporal force/torque data that the user is hammering with the tool, the neural network further determines whether the tool is hammering through bone or is hammering through tissue, wherein when the tool is determined to be hammering through tissue, the control mode is a first stiffness mode wherein the tool guide has a first stiffness, and wherein when the tool is determined to be hammering through bone, the control mode is a second stiffness mode wherein the tool guide has a second stiffness, wherein the second stiffness is less than the first stiffness.

In some embodiments, the method further comprises providing an alert to the user when the control mode is changed.

In some embodiments, the method further comprises: receiving auxiliary data comprising at least one of video data, image data, audio data, surgical plan data, diagnostic plan data and robot vibration data; and determining the current intention of the user or the state of the collaborative procedure based on the temporal force/torque data and the auxiliary data.

In yet another aspect of the invention, a processing system is provided for controlling a robotic arm having one or more degrees of freedom of control, wherein the robotic arm includes an instrument interface. The processing system comprises: a processor;

and memory having stored therein instructions. When executed by the processor, the instructions cause the processor to: receive temporal force/torque data, wherein the temporal force/torque data represents forces at the instrument interface over time during a collaborative procedure with a user, analyze the temporal force/torque data to determine at least one of a current intention of the user and a state of the collaborative procedure, and cause the robotic arm to be controlled in a control mode which is predefined for the determined current intention of the user or state of the collaborative procedure, wherein the control mode sets the at least one robot control parameter.

In some embodiments, the instrument interface comprises a tool guide which is configured to be interfaced with a tool which can be manipulated by the user during the collaborative procedure, and the forces comprise at least one of: (1) forces exerted indirectly on the tool guide by the user during user manipulation of the tool; (2) forces applied directly to the tool guide by the user; (3) forces from an environment of the robot; and (4) forces generated by the tool.

In some embodiments, the instructions further cause the processor to analyze the temporal force/torque data to identify a command provided by the user to the system to instruct the system to switch the control mode to a predefined mode.

In some embodiments, the at least one robot control parameter controls a rendered stiffness of the tool guide against the forces applied in at least one direction.

### BRIEF DESCRIPTION OF THE DRAWINGS

FIG. 1 illustrates an example of a surgical operating room in which a surgeon operates with a collaborative robot in a simulated spine fusion procedure.

FIG. 2 illustrates one example embodiment of a collaborative robot tool guide with force sensing.

FIG. 3 illustrates an example embodiment of a collaborative robot.

FIG. 4 illustrates is a block diagram illustrating an example embodiment of a processor and associated memory according to embodiments of the disclosure.

FIG. 5 illustrates force/torque profiles for hammering and drilling at different stages of a collaborative surgical intervention.

FIG. 6 illustrates an example of an arrangement for classifying events during a collaborative procedure based on force/torque data and robot data which maps a detected robot state.

FIG. 7 illustrates a first example embodiment of a control flow for automatically switching control modes of a collaborative robot based on force/torque state detection by the collaborative robot.

FIG. 8 illustrates a second example embodiment of a control flow for automatically switching control modes of a collaborative robot based on force/torque state detection by the collaborative robot.

FIG. 9 illustrates a flowchart of an example embodiment of a method of controlling a collaborative robot based on force/torque state detection by the collaborative robot.

### DETAILED DESCRIPTION

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

In particular, in order to illustrate the principles of the present invention, various systems are described in the context of robot-guided surgery, for example spinal fusion surgery. However, it will be understood that this is for the purposes of illustrating a concrete example of a collaborative robot and a method of operating a collaborative robot. More broadly, aspects of a collaborative robot and a method of operating a collaborative robot as disclosed herein may be applied in a variety of other contexts and settings. Accordingly, the invention is to be understood to be defined by the claims and not limited by details of specific embodiments described herein, unless those details are recited in the claims themselves.

Herein, when something is said to be “approximately” or “about” a certain value, it means within 10% of that value.

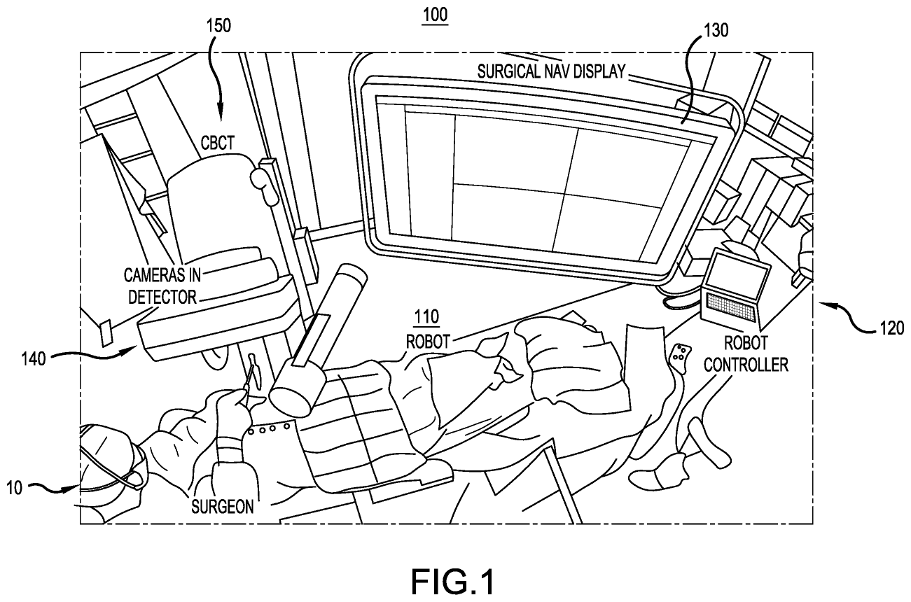

FIG. 1 illustrates an example of a surgical operating room 100 in which a surgeon operates with a collaborative robot 110 in a simulated robot-guided spinal fusion surgical procedure. Also shown in FIG. 1 are a robot controller 120, a surgical navigation display 130, cameras 140 and a cone beam computed tomography (CBCT) apparatus to assistant surgeon 10 in performing the robot-guided spinal fusion surgery procedure. Here, robot 110 is used to assist surgeon 10 in precise creation of a hole inside the pedicle (section of a vertebrae) along a planned trajectory. After the hole is created in multiple pedicles using a needle or a drill, surgeon 10 places a screw inside these pilot holes and fixes the adjacent screws with rods to fuse multiple vertebrae in a desired configuration. Currently, robot behaviors or modes are changed manually by the robot user or another human assistant, which is inefficient in total time and delays, and disrupts the workflow. In some cases, the human operator may not even know that the robot mode should be changed in time to be useful. Such changes may include, for example, changing robot compliance based on type of task being performed: e.g., drilling vs hammering, or changing safety zones (tool angles/locations) based on the type of tissue the instrument is going through.

Current approaches to managing this situation include threshold based event/state detection, but these are not robust and specific enough to discern the complexity and verity of signals that are correlated with a particular event or state of the procedure. The same goes for Fourier space analysis techniques. Furthermore, the mode changing has to be intuitive and transparent, so there exists a need communicate the type of behavior that is selected.

To address some or all of these needs, the present inventor has conceived of collaborative robot, and a control method for a collaborative robot, that utilize force sensing of the tool interaction forces to automatically alter robot behavior based on the state of the robot and the related dynamic force information sensed at the tool interface.

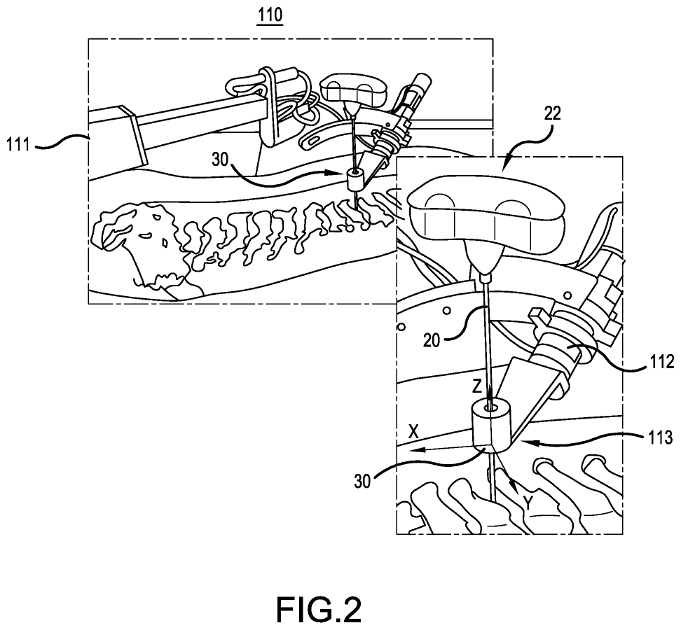

FIG. 2 illustrates one example embodiment of a collaborative robot 110 and an associated tool guide 30 with force sensing. As illustrated in FIG. 2, collaborative robot 110 includes a robot arm 111, with an instrument interface comprising a tool guide 30 disposed at an end effector 113 of robot arm 111. Here tool guide 30 may have a cylindrical shape, and a tool or instrument 20 (e.g., a drill, a needle, etc.), having a handle 22, passes through an opening in tool guide 30 for use by a surgeon during a surgical procedure (e.g., a spinal fusion surgical procedure).

Collaborative robot 110 also includes a force/torque sensor 112 which senses forces applied at or to tool guide 30 by a user during operation, e.g., forces applied indirectly by surgeon 10 while manipulating tool 20 in tool guide 30 during a spinal fusion surgical procedure as illustrated in FIG. 1, and/or forces which might be applied directly to tool guide 30 by the user or surgeon 10 as a form of command, as discussed in more detail below. In some cases, force/torque sensor may also sense forces from an environment of the robot and/or forces generated by tool or instrument 20. An example of a suitable force/torque sensor 112 is the Nano25 force/torque sensor—a six-axis transducer from ATI Industrial Automation, Inc.

Beneficially, collaborative robot 110 may be directly controlled by the user (e.g., surgeon 10) pushing on tool guide 30. Surgeon 10 may adjust collaborative robot 110 position using a hand-over-hand control (also known as “force control” or “admittance control”). Collaborative robot 110 may also function as a smart tool guide, precisely moving cylindrical tool guide 40 to a planned location and posture or pose for a planned trajectory, and holding that position while surgeon 10 engages instrument or tool 20 (e.g., a needle) inside tool guide 30 with the pedicle by either hammering or drilling.

As described in greater detail below, the admittance control method (using a signal from force/torque sensor 112) also allows for adjusting the compliance of collaborative robot 110, and more specifically end effector 113 and tool guide 30, independently in each degree of freedom (DOF), e.g., very stiff in Cartesian rotation but compliant in Cartesian translation.

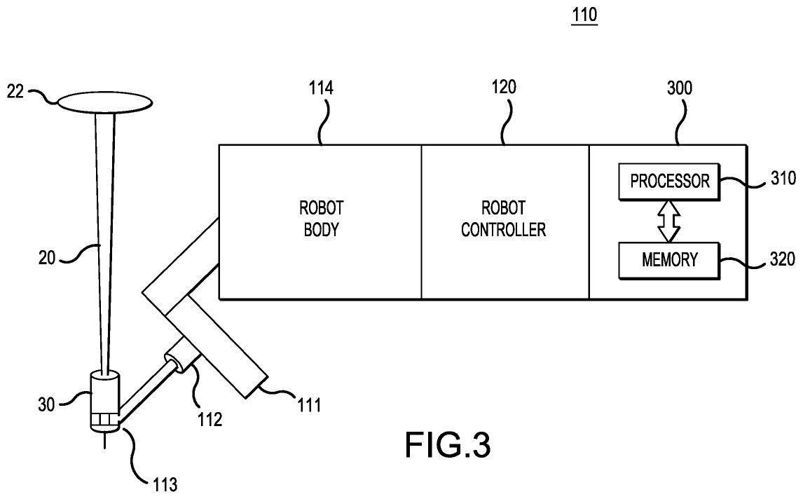

FIG. 3 illustrates a more general example embodiment of a collaborative robot 110.

Collaborative robot 110 includes a robot body 114 and robot arm 111 extending from robot body 114 with an instrument interface comprising a tool guide 30 held by end effector 113 disposed at the end of robot arm 111. End effector 113 may comprise a grasping mechanism for grasping and holding tool guide 30. FIG. 3 shows tool 20 passing through an opening in cylindrical tool guide 30, and having a handle 22 which may be manipulated by a user (e.g., surgeon) for performing a desired collaborative procedure.

Collaborative robot 110 also includes a robot controller 120 and a system controller 300. Robot controller 120 may comprise one or more processors, memory, actuators, motors, etc. for effecting movement of collaborative robot 110, and in particular movement and orientation of the instrument interface comprising tool guide 30. As illustrated in FIG. 3, system controller 300 may comprise one or more processors 310 and associated memory(ies) 320.

In some embodiments, robot controller 120 may be integrated with robot body 140. In other embodiments, some or all of the components of robot controller 120 may be provided separate from robot body 140, for example as a laptop computer, or other device which may include a display and a graphical user interface. In some embodiments, system controller 300 may be integrated with robot body 140. In other embodiments, some or all of the components of system controller 300 may be provided separate from robot body 140. In some embodiments, one or more processors or memories of system control 300 may be shared with robot controller 120. Many different partitions and configurations of robot body 140, robot controller 120, and system controller 300 are envisioned.

Robot controller 120 and system controller 300 are described in greater detail below.

Robot arm 111 may have one or more joints which each may have up to six degrees of freedom—for example translation along any combination of mutually orthogonal x, y and z axes, as well as rotation about the x, y and z axes (also referred to as yaw, pitch and roll). On the other hand, some or all of the joints of robot arm 111 may have less than six degrees of freedom. Movement of the joints in any or all degrees of freedom may be performed in response to control signals provided by robot controller 120. In some embodiments, motors, actuators, and/or other mechanisms for controlling one or more joints of robot arm 111 may be included in robot controller 120.

Collaborative robot 110 further includes force/torque sensor 112 which senses forces applied to or at the instrument interface, e.g., forces applied to tool guide 30 by tool disposed within tool guide 30 while tool 20 is being manipulated by surgeon 10 during a spinal fusion surgical procedure as illustrated in FIG. 1. In some embodiments, collaborative robot may comprise a plurality of force/torque sensors 112.

Robot controller 120 may control robot 110 in part in response to one or more control signals received from system controller 300 as described in greater detail below. In turn, system controller 300 may output one or more control signals to robot controller 120 in response to one or more signals received from force/torque sensor 112. In particular, system controller 300 receive temporal force/torque data, wherein the temporal force/torque data represents the forces applied to or at the instrument interface comprising tool guide 30 over time and which are sensed by force/torque sensor 112 during a collaborative procedure by a user. As described below, system 300 may be configured to interpret the signal(s) from force/torque sensor 112 to ascertain an intention and/or a command of the user of collaborative robot 110, and to control collaborative robot 110 to act in accordance with the user's intention and/or command, as expressed by the force/torque sensed by force/torque sensor 112.

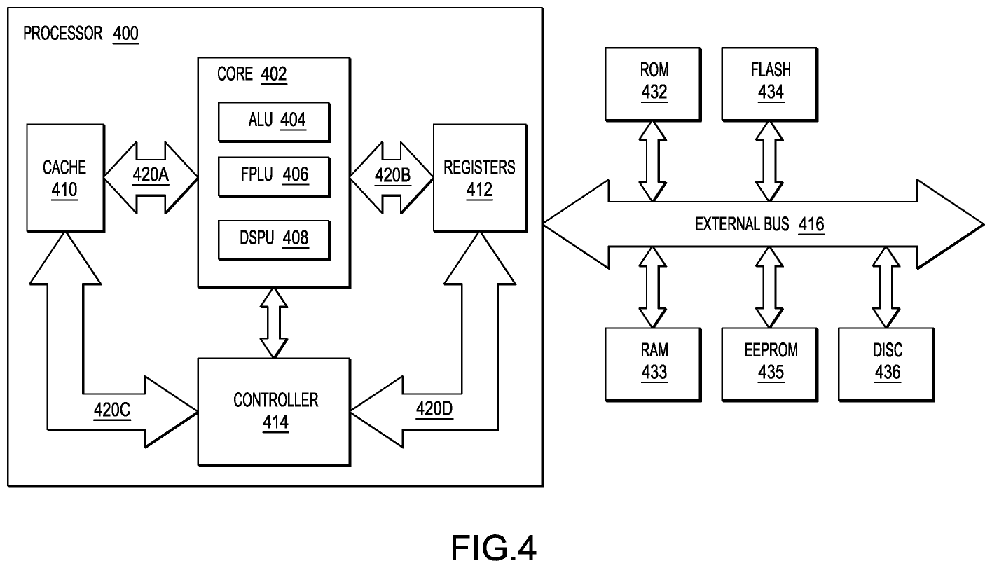

FIG. 4 illustrates is a block diagram illustrating an example embodiment of a processor 400 and associated memory 450 according to embodiments of the disclosure.

Processor 400 may be used to implement one or more processors described herein, for example, processor 310 shown in FIG. 3. Processor 400 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

Processor 400 may include one or more cores 402. Core 402 may include one or more arithmetic logic units (ALU) 404. In some embodiments, core 402 may include a floating point logic unit (FPLU) 406 and/or a digital signal processing unit (DSPU) 408 in addition to or instead of ALU 404.

Processor 400 may include one or more registers 412 communicatively coupled to core 402. Registers 412 may be implemented using dedicated logic gate circuits (e.g., flip-flops) and/or any memory technology. In some embodiments registers 412 may be implemented using static memory. Registers 412 may provide data, instructions and addresses to core 402.

In some embodiments, processor 400 may include one or more levels of cache memory 410 communicatively coupled to core 402. Cache memory 410 may provide computer-readable instructions to core 402 for execution. Cache memory 410 may provide data for processing by core 402. In some embodiments, the computer-readable instructions may have been provided to cache memory 410 by a local memory, for example, local memory attached to the external bus 416. Cache memory 410 may be implemented with any suitable cache memory type, for example, metal-oxide semiconductor (MOS) memory such as static random access memory (SRAM), dynamic random access memory (DRAM), and/or any other suitable memory technology.

Processor 400 may include a controller 414, which may control input to the processor 400 from other processors and/or components included in a system (e.g., force/torque sensor 112 in FIG. 3) and/or outputs from processor 400 to other processors and/or components included in the system (e.g., robot controller 120 in FIG. 3). Controller 414 may control the data paths in the ALU 404, FPLU 406 and/or DSPU 408. Controller 414 may be implemented as one or more state machines, data paths and/or dedicated control logic. The gates of controller 414 may be implemented as standalone gates, FPGA, ASIC or any other suitable technology.

Registers 412 and cache 410 may communicate with controller 414 and core 402 via internal connections 420A, 420B, 420C and 420D. Internal connections may be implemented as a bus, multiplexor, crossbar switch, and/or any other suitable connection technology.

Inputs and outputs for processor 400 may be provided via bus 416, which may include one or more conductive lines. Bus 416 may be communicatively coupled to one or more components of processor 400, for example controller 414, cache 410, and/or register 412. Bus 416 may be coupled to one or more components of the system, such as robot controller 120 mentioned previously.

Bus 416 may be coupled to one or more external memories. The external memories may include Read Only Memory (ROM) 432. ROM 432 may be a masked ROM, Electronically Programmable Read Only Memory (EPROM) or any other suitable technology. The external memory(ies) may include Random Access Memory (RAM) 433. RAM 433 may be a static RAM, battery backed up static RAM, Dynamic RAM (DRAM) or any other suitable technology. The external memory(ies) may include Electrically Erasable Programmable Read Only Memory (EEPROM) 435. The external memory(ies) may include Flash memory 434. The External memory(ies) may include a magnetic storage device such as disc 436. In some embodiments, the external memories may be included in a system, such as robot 110.

Collaborative robot 110 including force/torque sensor 112 was used to measure force/torque (FT) in a static tool guide holding mode during drilling and hammering into a pedicle, a common but extremely difficult task in spinal fusion.

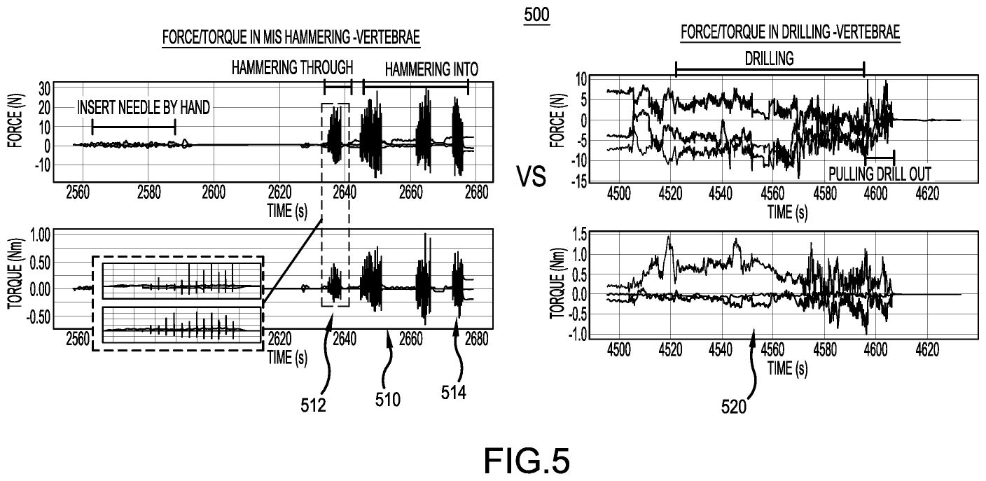

FIG. 5 illustrates force/torque profiles 500 for hammering and drilling at different stages of a collaborative surgical intervention.

FIG. 5 shows a first force/torque trace 510 representing the forces as a function of time which are applied to or at the instrument interface comprising tool guide 30 when a surgeon is performing a hammering operation with tool 20 which is disposed within tool guide 20. First torque trace 510 includes two distinct and recognizable temporal force/torque patterns, including a first temporal pattern 512 corresponding to the force/torque applied to the instrument interface comprising tool guide 30 during an operation or process of hammering through soft tissue, and a second temporal pattern 514 corresponding to the force/torque applied to the instrument interface comprising tool guide during an operation or process of hammering into bone. Second torque trace 520 shows a temporal pattern corresponding to the force/torque applied to the instrument interface comprising tool guide 30 during an operation or process of drilling through bone.

The force/torque traces of FIG. 5 depict clear differences in the sensed or measured temporal pattern of the force/torque applied to the instrument interface comprising tool guide 30 between hammering and drilling, and even between the types of tissue with which the instrument or tool is interacting.

As discussed in greater detail below, it is possible to configure system controller 300 to recognize these different patterns of temporal force/torque data and thereby ascertain what operation is being performed by the user (e.g., surgeon 10). The temporal force/torque data may be supplemented by knowledge of ordered operations that are expected to be performed during a particular surgical procedure, for example ordered operations that a surgeon should be expected to perform during a robot-guided spinal fusion surgery procedure. For example, an operation of hammering through tissue may be expected to be followed by an operation of hammering into bone, and then drilling into bone, etc. Such knowledge may be stored in a memory associated with system controller 300 and may be accessed by a processor of system controller while system controller 300 controls operation of collaborative robot 110 during a collaborative surgical procedure.

One system and method for detecting the state of the intervention (or user intention) during a collaborative procedure employs a recurrent neural network to consider a time series of force/torque measurement data, together with a current state of collaborative robot 110, such as a velocity of collaborative robot 110 resolved at the same location as the force/torque measurement data (e.g., resolved at tool guide 30). This type of network may be trained with data collected from multiple trials to improve its performance.

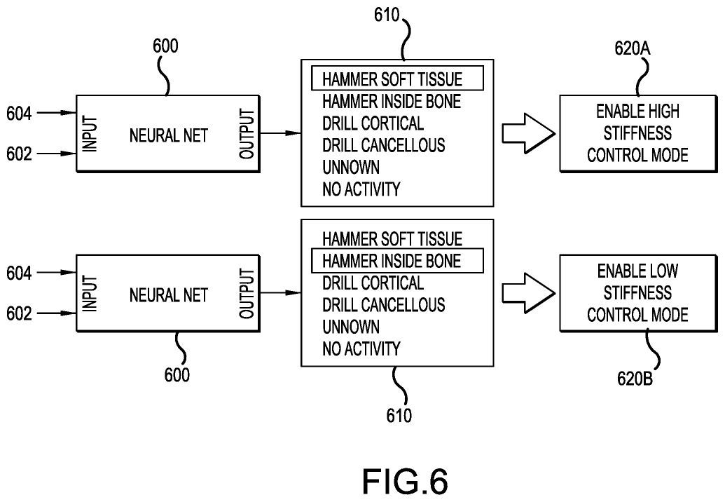

FIG. 6 illustrates an example of an arrangement for classifying events during a collaborative procedure based on force/torque data and robot data which maps a detected robot state.

FIG. 6 shows a neural network 600 receiving as inputs a robot state sequence 602 for collaborative robot 110 and temporal force/torque data 604 which represents the forces applied to the instrument interface comprising tool guide 30, e.g., forces applied to tool guide 30 by tool 20 disposed within tool guide 30 while tool 20 is manipulated by the user (e.g., surgeon 10)) over time during a collaborative procedure. Robot state sequence 602 is a temporal sequence of the robot states in which collaborative robot 110 has been previously operating up to the present, for example during a collaborative procedure. In response to temporal force/torque data 604 and robot state sequence 602, neural network 600 outputs a current robot state for collaborative robot 110 from the set of possible robot states 610 of collaborative robot 110.

Each of the possible robot states 610, in turn, corresponds to one or more control modes for collaborative robot 110. For example, as shown in FIG. 6, when neural network 600 ascertains that the current robot state for collaborative robot 110 is a “Hammer Soft Tissue” state, then it causes collaborative robot 110 to operate in an “Enable High Stiffness Control Mode” 620A. In contrast, when neural network 600 ascertains that the current robot state for collaborative robot 110 is a “Hammer Inside Bone” state, then it causes collaborative robot 110 to operate in an “Enable Low Stiffness Control Mode” 620B. In some cases, the relative stiffness may be reversed to keep the planned trajectory regardless of the anatomy geometry.

In some embodiments, neural network 600 may be implemented in system controller 300 by processor 310 executing a computer program defined by instructions stored in memory 320. Other embodiments realized with various combinations of hardware and/or firmware and/or software are possible.

In the example of FIG. 6, at any given time collaborative robot 110 may operate in any one of six defined robot states, including Hammer Soft Tissue, Hammer Inside Bone, Drill Cortical, Drill Cancellous, Unknown, and No Activity. Other robot states 610 are possible, such as a Moving state, a Skiving-Detected state, a Retraction state, an Inserting Instrument in Tool Guide state, a Gesture-Detected state (indicating a specific user control is desired), etc., depending on the collaborative procedure in which collaborative robot 110 is engaged.

Beneficially, each robot state 610 may be defined as a Cartesian velocity resolved at the instrument interface comprising tool guide 30 (at the end of end-effector 113). Velocity may be calculated from joint encoder values and a forward kinematic model for collaborative robot 110 and the robot's Jacobian, which relates the joint rates to the linear and angular velocity of end-effector 113.

Robot state 610 may also encompass, for example: a velocity from an external tracking system (optical tracker, electromagnetic tracker); torque on the motors for robot arm 111; the type of tool 20 used; the state of the tool (drill on or off, positional tracking, etc.); data from accelerometers on tool 20 or collaborative robot 110; etc.

Force/torque data may represent force/torque in one or more degrees of freedom. In general, the force/torque may be resolved at one of several different convenient locations. Beneficially, the force/torque may be resolved at tool guide 30 at the end of end effector 113. In general, temporal force/torque data 604 doesn't need to be preprocessed beyond basic noise reduction, and processing to resolve the force/torque at a particular location (e.g., at tool guide 30 at the end of end effector 113).

Beneficially, a method of robot state detection may use a data-driven model (e.g., via neural network 600) to continuously classify the robot state of the procedure or intervention (aka the class) based on a short period of data output from force/torque sensor 112 (i.e., temporal force/torque data 604), and the robot state 610. There are many potential model architectures that may be used to classify time-series data with multiple inputs. A beneficial model is the Long-Short Term Memory (LSTM) network because it is more stable than a typical Recurrent Neural Network (RNN). Other examples of possible networks include an Echo State Network, a convolutional neural network (CNN), a Convolutional LSTM, and hand-crafted networks using multiplayer perceptron, decision trees, logistic regression, etc.

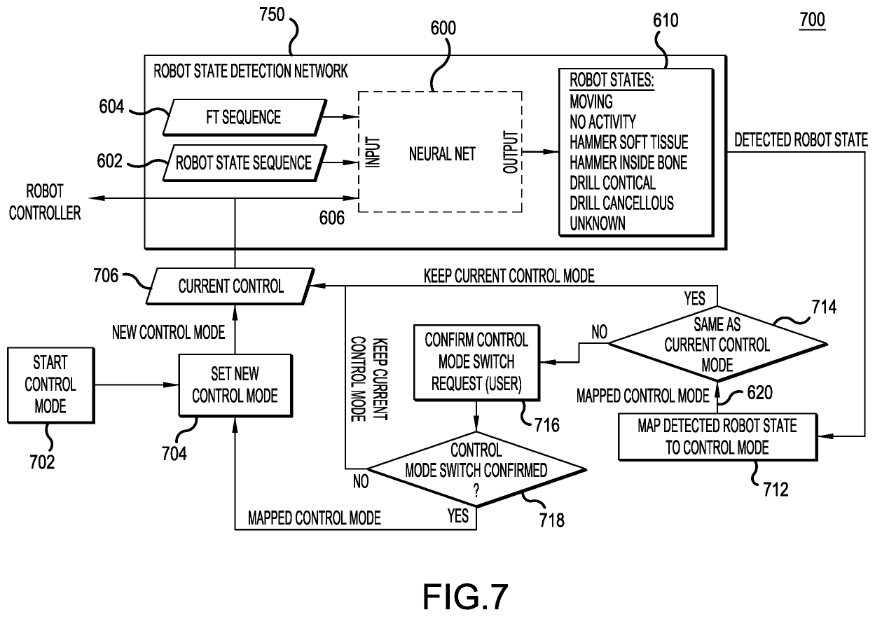

Beneficially, the data streams input into neural network 600 (which may include force/torque data (604), robot state (602), current control mode (see FIG. 7 below), etc.) are preprocessed (interpolated, downsampled, upsampled, etc.) to have the same period per data point (typically the period of the highest frequency data stream which is typically the temporal data from force torque sensor 112 (e.g., at 1 kHz, or a 1 ms period). In some embodiments, the temporal sliding window for neural network 600 may be set to be about three (3) seconds long to capture typical events such as drilling, reaming, pushing, pulling, screwing, and hammering, (e.g., hammering has a typical interval of about one (1) second), while short enough to be responsive for a given task. Each window shift (advancement) is a new sample to be classified, and in a training phase it has an associated robot state label. Windows with smaller sizes may be discarded.

In some embodiments, a single input sample may contain 12 features for each of N time steps (e.g., 36K individual features for 3 seconds at 1 kHz sampling) each: 3 for Force, 3 for Torque, 3 for XYZ robot linear velocity, 3 for robot angular velocity. In some embodiments, the output of the LSTM is the probability of each robot state for a given input window sample. In some embodiments, the model has two LSTM hidden layers followed by a dropout layer (to reduce overfitting), followed by a dense fully connected layer with common Rectified Linear Unit (“ReLU”) activation function, and the output layer with normalized exponential function (“softmax”) activation. LSTM and ReLU are building blocks of common deep learning models as would be understood by those of ordinary skill in the art. Loss function is categorical cross entropy and the model may be optimized using adaptive learning rate optimization algorithm (Adam) optimizer. Adam optimizer is a widely used optimizer for deep learning model as described in, e.g., Diederik P. Kingma et al., “Adam: A method for stochastic optimization,” 3RD INTERNATIONAL CONFERENCE ON LEARNING REPRESENTATIONS, (San Diego, 2015).

Beneficially, in training care is employed to balance examples of all the different robot states which are expected, especially the ones that are seldomly experienced (e.g., Drilling) compared to the most common robot state(s) (e.g., Inactive). This reduces the bias towards the common robot states. Techniques may include under-sampling the most common robot states and/or oversampling the rare robot states in training sequences. In addition, a cost-based classifier is used to penalize incorrectly classifying the robot state of interest while reducing the cost of correctly classifying the common robot state(s). This is especially useful in cases where infrequent events happen within in the temporal window (e.g., three hammer strokes with no activity vs continuous drilling for three seconds).

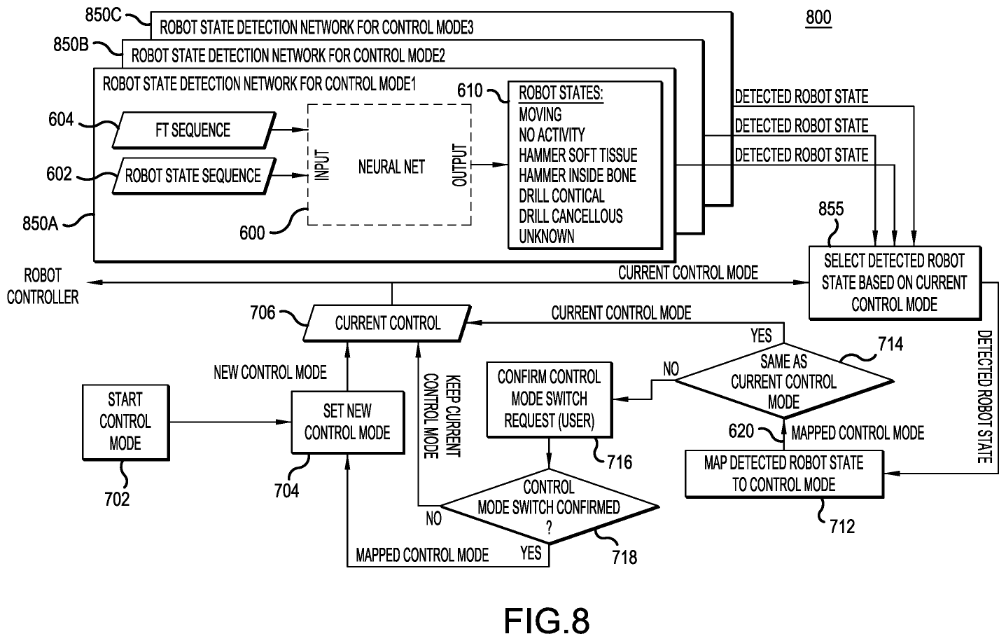

FIGS. 7 and 8 illustrate two different examples of control mode switching algorithms for a collaborative robot such as collaborative robot 110.

FIG. 7 illustrates a first example embodiment of a control flow 700 for automatically switching control modes of collaborative robot 110 based on force/torque state detection by collaborative robot 110. Control flow 700 may be implemented by system controller 300, and more specifically by processor 310 of system controller 300. Control flow 700 employs a single model (and corresponding single neural network 600) with a current mode input 606 to detect a robot state 610 during the current control mode 620. Neural network 600 implicitly considers the current control mode context in robot state detection.

Initially, in operation 702 system controller 300 selects a starting control mode, for example either in response to direct input from a user (e.g., surgeon 10) or as a preprogrammed initial control mode for collaborative robot 110 which may be determined for a specific collaborative procedure.

In operation 704, system controller 300 sets a current control mode 706 for collaborative robot 110, initially as the starting control mode. System controller 300 may provide one or more signals to robot controller 120 to indicate current control mode 706 and/or to cause robot controller 120 to control collaborative robot 110, and specifically robot arm 111, in accordance with current control mode 706. By setting the current mode, examples of which have been described above, system controller 300 may control one or more robot control parameters, including for example controlling an amount of rendered stiffness of tool guide 30 against forces applied to it in one or more of up to six degrees of freedom, (e.g., in at least one direction).

In some embodiments, system controller 300 may control other robot control parameters besides rendered stiffness at tool guide 30, such as position limits (trajectory constraints), dwell time (in a particular location), accelerations of robot arm 111, vibrations, drilling speeds (on/off) of tool 20, maximum and minimum velocities, etc.

Control flow 700 employs a robot state detection network 750 to ascertain or detect a robot state 610 of collaborative robot 110. State detection network 750 includes neural network 600, as described above, which receives as its inputs robot state sequence 602 for collaborative robot 110, temporal force/torque data 604, and the current control mode 606, and in response thereto selects a robot state 610 from among a plurality of possible robot states for collaborative robot 110.

An operation 712 maps the detected robot state 610, detected by robot state detection network 750, to a mapped control mode 620 for collaborative robot 110.

An operation 714 determines whether mapped control mode 620 is the same as the current control mode 706 for collaborative robot 110. If so, then current control mode 706 remains the same. If not, then current control mode 706 should be changed or switched to mapped control mode 620.

In some embodiments, in an operation 716, system controller 300 may alert a user to the fact that system controller 300 has a pending control mode switch request. System controller 300 may request that the user (e.g., surgeon 10) confirm or approve the control mode switch request. In some embodiments, operation 716 may only be executed for some specific procedures, but skipped for other procedures.

In some embodiments, a control mode switch request of operation 716 may be presented to the user via a user interface associated with system controller 300, for example visually via a display device (e.g., display device 130), or audibly (e.g., verbally) via a speaker, etc.

In those embodiments or procedures where the control mode switch request of operation 716 is executed, then in an operation 718 system controller determines whether or not the user (e.g., surgeon 10) confirms or approves the control mode switch request. The user or surgeon may confirm or approve (or, conversely deny or disapprove) the control mode switch request in any of a variety of ways. Examples include:
The user or surgeon may respond by clicking a pedal or a button of a user interface to confirm the change of control mode.
Voice recognition may be used to confirm the user's approval or acceptance of a change of control mode.
The user may approve or accept a change of control mode via hand/body gesturing, which may be detected using visual or depth tracking camera and provided as a supplemental or auxiliary data input to system controller 300.

In some cases confirmation-less control mode switching may be acceptable, and these cases may be mixed with some cases which require user input. Thus operations 716 and 718 may be optional. In those cases, a simple audible effect that indicates to the user or surgeon which mode has been entered could be sufficient. The user or surgeon may then cancel or stop robot motion if this is not a desirable mode. Beneficially, the current detected robot state, and control mode may be clearly communicated to the user or surgeon using audio-visual means, such as a digital display, LED lights on the robot, voice feedback describing the system as it is sensing and changing, etc.

If the control mode switch request is not approved, then current control mode 706 is maintained.

On the other hand, control mode switch request is approved, or if operations 716 and 718 are omitted, then operation 704 is repeated to set mapped mode 620 as a new current control mode 706 for collaborative robot 110. The new current control mode 706 is provided to input 606 of neural network 600 and as one or more output signals to robot controller 120.

FIG. 8 illustrates a second example embodiment of a control flow 800 for automatically switching control modes of a collaborative robot based on force/torque state detection by the collaborative robot. Control flow 800 may be implemented by system controller 300, and more specifically by processor 310 of system controller 300.

For the sake of brevity, descriptions of operations and flow paths in control mode 800 which are the same as those in control mode 700 will not be repeated.

In contrast to control flow 700, control flow 800 employs a plurality of robot state detection networks 850A, 850B, 850C, etc. for detecting a robot state, one for each control mode selected upon a control mode switch event. Each of the robot state detection networks 850A, 850B, 850C, etc. implements a corresponding model for robot state detection and outputs a corresponding detected robot state. In control flow 800, the detected robot state which is output from one of the plurality of models (and its corresponding neural network 600) is explicitly selected for each control mode in operation 855.

In some embodiments, a handcrafted state machine layer may be added to prevent false positives and negatives, to add a temporal filter, and to consider the procedure plan or longer term state transitions. For example, in case of pedicle drilling, it is unlikely that the physician will hammer the drill bit after drilling is being performed, and a higher level state machine may be included in the control flow for detection of this inconsistency. An error may be communicated that collaborative robot 110 is not being used properly or that the procedure is not being followed.

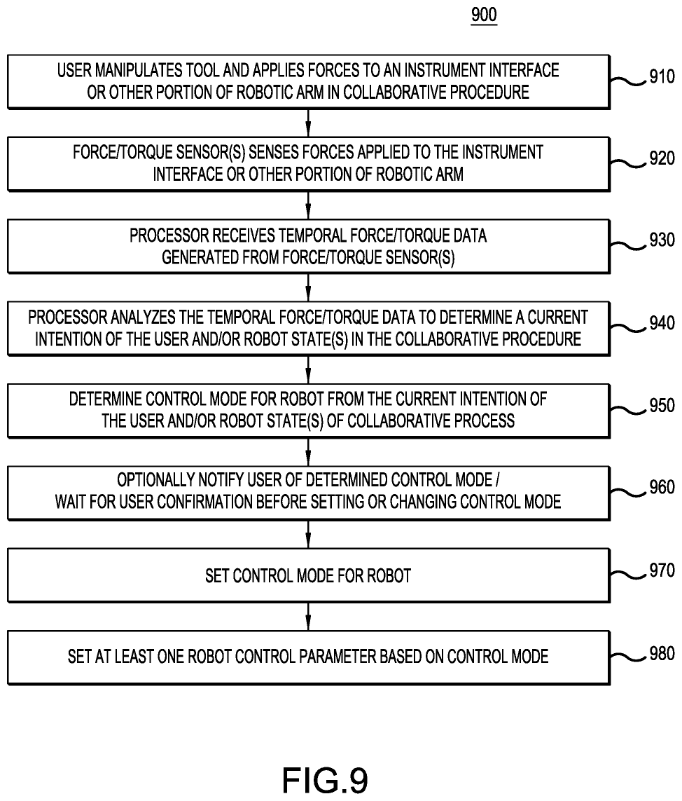

FIG. 9 illustrates a flowchart of an example embodiment of a method 900 of controlling a collaborative robot (e.g., collaborative robot 110) based on force/torque state detection by collaborative robot 110 during a procedure or intervention.

In an operation 910, a user (e.g., surgeon 10) manipulates an instrument or tool (e.g., tool 20) which applies forces to an instrument interface (e.g., tool guide 30) or other portion of robotic arm 111 in a collaborative procedure (e.g., a spinal fusion surgical procedure).

In an operation 920, force/torque sensor(s) 112 senses forces applied to the instrument interface or other portion of robotic arm 111, for example at tool guide 30.

In an operation 930, processor 310 of system controller 300 receives temporal force/torque data 604 generated from force/torque sensor(s) 112.

In an operation 940, processor 310 analyzes temporal force/torque data 604 to determine a current intention of the user and/or one or more robot state(s) during the collaborative procedure.

In an operation 950, system controller 400 determines a control mode for collaborative robot 110 from the current intention of the user, and/or a current robot state and/or past robot state(s) during the collaborative procedure.

In an operation 960, system controller 300 notifies the user of the determined control mode to which the collaborative robot should be set, and waits for user confirmation before setting or changing the current control mode to the determined control mode.

In an operation 970, system controller 300 sets the control mode for collaborative robot 110.

In an operation 980, system controller 300 sets one or more robot control parameters, based on the current control mode. The one or more robot control parameters may control, for example, an amount of rendered stiffness for tool guide 30 in one or more of up to six degrees of freedom. In some embodiments, system controller 300 may control other operating parameters besides rendered stiffness at tool guide 30, such as position limits (trajectory constraints), dwell time (in a particular location), etc.

Many variations of the embodiments described above are envisioned.

For example, in the basic case described above, force/torque sensor 112 is located between the main robot body 114 and tool guide 30. However in some embodiments, force/torque sensor 112 may be located near tool guide 30 or integrated into robot body 114. Beneficially, a six-degrees-of-freedom force/torque sensing technique may be employed. Torque measurements on the joints of robot arm 111 may also provide basic information on force/torques resolved at tool guide 30. The forces may be resolved at tool guide 30 or at the estimated or measured location of the tip of tool 20.

In some robot/sensor configurations, system controller 300 may discern a user's applied input force/torque from the force/torque exerted by the environment on the instrument (e.g., force/torque sensor integrated on instrument tip, and another force/torque sensor on the tool guide). For example, environmental forces (e.g., tissue pushing on the tool) may be a primary source of feedback information for a data model to ascertain whether a tool is going through soft tissue or through bone. That is, the environment forces include the result of the anatomy responding to the stimulus provided by the user and the robot through the tool.

In some embodiments, system controller 300 may consider different inputs for detecting the robot state during a collaborative procedure or interventions. Examples of such inputs may include:
Frequency domain of force/torque data
Frequency domain of Velocity/Acceleration data
Current robot state
Estimated position to target
Type of procedure
Estimated bone type
Estimated tissue type at instrument tip (from navigation)
Robot stiffness
Robot control mode
Computed tomography data
Magnetic resonance imaging data

Each of these data inputs have different behavior and may be considered depending on the desired focus of the collaborative robot 110.

In some embodiments, system controller 300 may receive supplemental or auxiliary data input to help discern the context (search space) to improve robot state detection. Such data may include one or more of the following: video data, diagnostic data, image data, audio data, surgical plan data, time data, robot vibration data, etc. System controller 300 may be configured to determine the current intention of the user (surgeon) or the state of the collaborative procedure based on the temporal force/torque data and the auxiliary data.

In some embodiments, a user (e.g., surgeon 10) may also apply force/torque to collaborative robot 110 in a very particular way to engage a specific control mode. For example, system controller 300 of collaborative robot 110 may be configured to recognize when the user applies circular force on tool guide 30 (via the instrument or tool 209 in tool guide 20, or by applying the force directly to tool guide 30), an in response thereto system controller 300 may place collaborative robot 110 into a canonical force control mode (e.g., admittance controller that allows the operator to move the robot by applying a force to it in the desired direction). In other words, a processor of system controller 300 may be configured to analyze temporal force/torque data 604 to identify a command provided by the user to system controller 300 to instruct system controller 300 to switch the control mode for collaborative robot 110 into a predefined control mode. Some other example of specific pressure actions of a user which may be interpreted as control mode commands may include:
The user or surgeon presses down and up 3 times—translation only mode.
The user makes a circular motion pressing up twice—insertion only mode.
The user applies pressure in a specific sequence (e.g., left, right, up, down)—select next planned trajectory.

Many other examples of specific commands for corresponding control modes may be employed.

In some embodiments, force/torque sensing as described above may also be supplemented or substituted with vibration sensing of the robot itself (e.g., via an accelerometer). Events like hammering and drilling induce vibrations in the robot structure which may be detected away from the robot tool effector and used in the same way as described above.

Various embodiments may combine the variations described above.

While preferred embodiments are disclosed in detail herein, many other variations are possible which remain within the concept and scope of the invention. Such variations would become clear to one of ordinary skill in the art after inspection of the specification, drawings and claims herein. The invention therefore is not to be restricted except within the scope of the appended claims.

What is claimed is:

1. A system, comprising:
a robotic arm having one or more degrees of freedom of control, wherein the robotic arm includes an instrument interface;
at least one force/torque sensor configured to sense forces at the instrument interface;
a robot controller configured to control the robotic arm to move the instrument interface to a determined position and to control at least one robot control parameter; and
a system controller configured to:
receive temporal force/torque data, wherein the temporal force/torque data represents the forces at the instrument interface over time, sensed by the at least one force/torque sensor during a collaborative procedure with a user,
analyze the temporal force/torque data to determine at least one of a current intention of the user and a state of the collaborative procedure, and
cause the robot controller to control the robotic arm in a control mode which is predefined for the determined current intention of the user or state of the collaborative procedure, wherein the control mode determines the at least one robot control parameter.

2. The system of claim 1, wherein the instrument interface comprises a tool guide which is configured to be interfaced with a tool which can be manipulated by the user during the collaborative procedure, and wherein the forces comprise at least one of: (1) forces applied indirectly to the tool guide during user manipulation of the tool; (2) forces applied directly to the tool guide by the user; (3) forces from an environment of the robot; and (4) forces generated by the tool.

3. The system of claim 2, wherein the system controller is configured to apply the temporal force/torque data to a neural network to determine the current intention of the user or the state of the collaborative procedure.

4. The system of claim 3, wherein the neural network is configured to determine from the temporal force/torque data when the user is drilling with the tool, and is further configured to determine from the temporal force/torque data when the user is hammering with the tool.

5. The system of claim 4, wherein the at least one robot control parameter controls a rendered stiffness of the tool guide against the forces applied in at least one direction.

6. The system of claim 5, wherein when the neural network determines from the temporal force/torque data that the user is hammering with the tool, the neural network further determines whether the tool is hammering through bone or is hammering through tissue, wherein when the tool is determined to be hammering through tissue, the control mode is a first stiffness mode wherein the robot controller controls the tool guide to have a first stiffness, and wherein when the tool is determined to be hammering through bone, the control mode is a second stiffness mode wherein the robot controller controls the tool guide to have a second stiffness, wherein the second stiffness is less than the first stiffness.

7. The system of claim 1, wherein the system provides an alert to the user when the system changes the control mode.

8. The system of claim 1, wherein the system controller is further configured to receive auxiliary data comprising at least one of video data, image data, audio data, surgical plan data, diagnostic plan data and robot vibration data, and is still further configured to determine the current intention of the user or the state of the collaborative procedure based on the temporal force/torque data and the auxiliary data.

9. A method of operating a robotic arm having one or more degrees of freedom of control, wherein the robotic arm includes an instrument interface, the method comprising:
receiving temporal force/torque data, wherein the temporal force/torque data represents forces at the instrument interface over time, sensed by at least one force/torque sensor during a collaborative procedure with a user;
analyzing the temporal force/torque data to determine at least one of a current intention of the user and a state of the collaborative procedure; and
controlling the robotic arm in a control mode which is predefined for the determined current intention of the user or state of the collaborative procedure, wherein the control mode determines at least one robot control parameter.

10. The method of claim 9, wherein the instrument interface comprises a tool guide which is configured to be interfaced with a tool which can be manipulated by the user during the collaborative procedure, and wherein the force/torque sensor measures at least one of: (1) forces exerted indirectly on the tool guide by the user during user manipulation of the tool; (2) forces applied directly to the tool guide by the user; (3) forces from an environment of the robot; and (4) forces generated by the tool.

11. The method of claim 10, wherein analyzing the temporal force/torque data to determine at least one of the current intention of the user and the state of the collaborative procedure comprises applying the temporal force/torque data to a neural network to determine the current intention of the user or the state of the collaborative procedure.

12. The method of claim 11, wherein the neural network determines from the temporal force/torque data when the user is drilling with the tool, and further determines from the temporal force/torque data when the user is hammering with the tool.

13. The method of claim 12, wherein the at least one robot control parameter controls a rendered stiffness of the tool guide against the forces applied in at least one direction.

14. The method of claim 13, wherein when the neural network determines from the temporal force/torque data that the user is hammering with the tool, the neural network further determines whether the tool is hammering through bone or is hammering through tissue, wherein when the tool is determined to be hammering through tissue, the control mode is a first stiffness mode wherein the tool guide has a first stiffness, and wherein when the tool is determined to be hammering through bone, the control mode is a second stiffness mode wherein the tool guide has a second stiffness, wherein the second stiffness is less than the first stiffness.

15. The method of claim 9, further comprising providing an alert to the user when the control mode is changed.

16. The method of claim 9, further comprising:
receiving auxiliary data comprising at least one of video data, image data, audio data, surgical plan data, diagnostic plan data and robot vibration data; and
determining the current intention of the user or the state of the collaborative procedure based on the temporal force/torque data and the auxiliary data.

17. A processing system for controlling a robotic arm having one or more degrees of freedom of control, wherein the robotic arm includes an instrument interface, the processing system comprising:
a processor; and
memory having stored therein instructions which, when executed by the processor, cause the processor to:
receive temporal force/torque data, wherein the temporal force/torque data represents forces at the instrument interface over time during a collaborative procedure with a user,
analyze the temporal force/torque data to determine at least one of a current intention of the user and a state of the collaborative procedure, and
cause the robotic arm to be controlled in a control mode which is predefined for the determined current intention of the user or state of the collaborative procedure, wherein the control mode sets the at least one robot control parameter.

18. The system of claim 17, wherein the instrument interface comprises a tool guide which is configured to be interfaced with a tool which can be manipulated by the user during the collaborative procedure, and wherein the forces comprise at least one of: (1) forces exerted indirectly on the tool guide by the user during user manipulation of the tool; (2) forces applied directly to the tool guide by the user; (3) forces from an environment of the robot; and (4) forces generated by the tool.

19. The system of claim 18, wherein the instructions further cause the processor to analyze the temporal force/torque data to identify a command provided by the user to the system to instruct the system to switch the control mode to a predefined mode.

20. The system of claim 18, wherein the at least one robot control parameter controls a rendered stiffness of the tool guide against the forces applied in at least one direction.
